(19) United States
(12) Patent Application Publication
(10) Pub. No.: US 2002/0173489 A1
(43) Pub. Date: Nov. 21, 2002

(54) COMPOSITIONS CONTAINING A MIXTURE OF PHOSPHOROUS COMPOUNDS AND ALKYLGLYCEROLS

(76) Inventor: HANSJORG EIBL, BOVENDEN (DE)

Correspondence Address: FULBRIGHT & JAWORSKI, LLP, 666 FIFTH AVE, NEW YORK, NY, 10103-3198, US

(21) Appl. No.: 09/259,526

(22) Filed: Feb. 26, 1999

Related U.S. Application Data
(63) Continuation-in-part of application No. 08/458,232, filed on Jun. 2, 1995, now Pat. No. 5,916,884.
Application No. 08/458,232 is a continuation-in-part of application No. 07/912,554, filed on Jul. 13, 1992, now abandoned.
Application No. 07/912,554 is a continuation-in-part of application No. 07/517,440, filed on May 1, 1990, now abandoned.
Application No. 07/517,440 is a continuation of application No. 07/096,918, filed on Oct. 2, 1987, now abandoned.

(51) Int. Cl. A61K 31/685; A61K 31/66

(52) U.S. Cl. 514/77; 514/144; 514/129; 514/134; 514/137; 514/148

(57) ABSTRACT

The object of the invention is to provide a medicament which is suitable for the treatment of tumors, such as via topical administration.

Another object of the invention is to provide a medicament which, in general, can also be used in other forms of administration, such as in intravenous form, which combines good effectiveness against tumors, lower toxicity and which can, therefore, be generally used in tumor therapy.

### CROSS-REFERENCE TO RELATED APPLICATIONS

This application is a continuation-in-part of Ser. No. 08/458,232, filed Jun. 2, 1995, now U.S. Pat. No. ______, which is a continuation-in-part of Ser. No. 07/912,554, filed Jul. 13, 1992, which in turn is a continuation-in-part of Ser. No. 07/517,440, filed May 1, 1990, now abandoned; which in turn is a continuation of application Ser. No. 07/096,918, filed Oct. 2, 1987, now abandoned.

### FIELD OF THE INVENTION

This invention relates to novel therapeutic compositions having activity as a biological response modifier, which are especially suitable for the treatment of tumors.

### BACKGROUND OF THE INVENTION

European Application 108 565 concern compounds of the formula

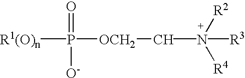

wherein R¹ is an aliphatic C₈-C₃₀-hydrocarbon radical and R², R³ and R⁴ are hydrogen, lower alkyl, the group

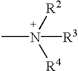

or a cyclic ammonium radical, and n=0. For these compounds, it is stated that they inhibit the proliferation oftumor cells and prolong the life span of warm-blooded animals which have such tumors; furthermore, an anti-fungal action is mentioned.

It is known that heretofore no medicament for the treatment of tumors, especially of malignant tumors, was available which was satisfactory in all respects. Thus, for example, for the topical treatment of skin metastases in patients with metastasizing tumors, at present only 5-fluorouracil is available. Further developments of other cytostatics have heretofore not progressed to clinical maturity for this manner of administration. On the other hand, from a clinical point of view, such a concept of palliative therapeutic use is especially desirable since alternative concepts of treatment, such as surgical measures, radiation therapy and systemic chemotherapy, constitute comparatively aggressive therapy modalities. Furthermore, a considerable number of patients are available as potential treatment candidates for such a topical treatment. Thus, the proportion of mammary carcinoma patients who display a skin attack amounts to about 25 to 35%.

The prerequisites for topical treatment on the part of the active material to be used are compatibility with the skin, cytotoxic effectiveness against tumor cells and sufficiently deep penetration.

### OBJECTS OF THE INVENTION

The object of the invention is to provide a medicament which is suitable for the treatment of tumors, such as via topical administration.

Another object of the invention is to provide a medicament which, in general, can also be used in other forms of administration, such as in intravenous form, which combines good effectiveness against tumors, lower toxicity and which can, therefore, be generally used in tumor therapy.

Other objects and advantages ofthe invention will become apparent as the description thereof proceeds.

### DESCRIPTION OF THE INVENTION

These objects are achieved via a medicament which contains as active material at least one compound of the formula

R-Y-P^{⊖}₂-X-R₁ (I or I′)

as defined below except hexadeclyphosphocholine, or aphysiologically acceptable salt hereof, either alone or in combination with conventional pharmacological additives and diluents. Preferred active materials, are oleylphosphocholine or hexadecylphosphoric acid -(N,N)-bis-(2-chloroethyl)-amide.

The invention includes all possible enantiomers and diastereoisomers of the compounds discussed supra. If the compounds are racemates, these can be resolved in any of the standard methods used therefor, for example by means of an optically active acid, into the optically active isomers. Preferably, however, enantiomeric or optionally diastereoisomeric starting materials are used, whereby the end product is obtained as a corresponding pure optically active or diastereoisomeric compound.

In the scope of the invention, R is preferably an alkyl group of a chain length described infra which is linked with the oxygen atom of the glycol residue via a terminal C-atom or also via a C-atom within the alkyl chain (for example via the C-atom 2 or C-atom 3 or another middle C-atom). This alkyl chain can be straight or branched. The alkyl chain R can contain one, two or three carbon double bonds or triple bonds, which can also be present mixed, and/or halogen substituents such as fluorine, chlorine or bromine. One to three of such halogen atoms can be present in the chain R, and these can be present on one or on different C-atoms of the radical R.

In especially preferred embodiments, R is a hydrocarbon residue 16-20 carbon atoms in length, preferably 17-19 carbon atoms in length, which is either saturated, or substituted with one or several double or triple bonds, or both. Double bonds are preferably arranged in syn configuration between C6 and C(ω-3), or at a terminus, i.e., at Cω-1. When two double bonds are present in the compound, these are preferably either two, isolated cis double bonds, the first of which is between C6 and C(ω-6), and the second of which is located between C10 and C(ω-3). Alternatively, there is a cis double bond between C6 and C(ω-6), and a terminal double bond at Cω-1. In especially preferred embodiments, R is a hexadecyl, heptadecyl, octadecyl, nonadecyl, eicosyl, cis-heptadec-8-enyl, cis-octadec-9-enyl (oleyl), cis-nonadec-10-enyl, or cis-eicos-11-enyl residue. With respect to the other moieties in the molecule Y and X are preferably oxygen, and R₁ is preferably CH₂CH₂N⁺R2R3R4, wherein R2, R3 and R4 are each methyl, ethyl, propyl, 2-hydroxyethyl, cyclopropyl, cyclobutyl, and cyclopentyl.

With respect to the above especially preferred definitions for R, compounds where both R2 & R3 are methyl, and R4 is methyl or ethyl are especially preferred.

Examples of halogen-substituted radicals R are: chlorohexadecyl, bromohexadecyl, fluorohexadecyl, 9,10-dibromo-octadecyl, 2,3-dibromooctadecyl, 15,16-dibromohexadecyl, and bromotetradecyl.

Examples of unsaturated radicals R include 9-octadecenyl radical (oleyl alcohol radical, in formula I or I′, R especially signifies this 9-octadecenyl radical), and 15-decadecenyl radical, and 9,12-octadecadienyl radical (linoleyl radical).

If more than one double or triple bond is present, these are conjugated.

Examples of saturated and unsubstituted radicals R are tetradecyl, hexadecyl and octadecyl.

If R₁ or R₂ signifies an unsubstituted alkyl group, this consists, e.g., of 1 to 6, and preferably 1-4 carbon atoms. If R₁ or R₂ signifies an unsaturated alkyl group, it preferably consists of 3 to 6 C-atoms, where, between the unsaturated function of such an unsaturated alkyl group and X, there must be at least one single C-C bond. C₃-C₆-alkylene groups are preferred. Examples of these are allyl, butenyl, pentenyl and hexenyl groups.

If R₁ or R₂ is substituted, straight-chained alkyl or alkenyl radicals are preferred. R₁, in this case preferably consists of 2-6 C-atoms, where the given substituents preferably stand in the w-position of the alkyl or alkenyl group R₁ or R₂; for example, the ethyl or straight chain propyl radical with one of the given substituents in w-position (that is, in 2-position in the case of ethyl and 3-position in the case of propyl) is preferred. If R₁ is a 2-tert.-butyloxycarbonylaminoethyl radical or a 2-tert.-butyloxycarbonylethyl radical, it is preferably the D- or L-form.

Amongst the substituents of R₁, the trialkylammoniumethyl radicals are preferred when X is an oxygen atom, where the trialkyl radicals preferably each consist of one, two or three C-atoms, preferably methyl groups. In this especially preferred embodiment form they are, in the case of formula I, phosphatidylcholine derivatives.

In the case of the C₃-C₈-cycloalkyl substituents, this 3-6 carbon atoms (for example, cyclopropyl to cyclohexyl) are preferred. In the case of the 2,3-dihydroxypropyl-(1) group the sn-1,2-dihydroxypropylamino-(3) structure or the sn-2,3-dihydroxypropylamino-(1) structure is preferred.

Of these compounds listed supra, the following are preferred: oleyl-phospho-(N,N,N-trimethyl)-propanolamine, oleylphospho-(N,N,N-trimethyl)-butanolamine, oleylphospho (N,N,N-trimethyl)-pentanolamine, oleylphosphoserine, oleylphosphoethanolamine, oleylphosphopropanolamine, oleylphosphobutanolamine, oleylphosphoglycerol, and hexadecylphospho-(N,N,N-trimethyl)-propanolamine.

The salts may be internal salts (for example, when R₁ is a trimethylammoniumalkyl group) or salts with physiologically acceptable cations. The compounds of this invention can be present as internal salts, for example when R₁ contains an amino group. If no internal salts are present or R₁ does not contain a basic group, the negative charge of the phosphoric acid group is satisfied by aphysiologically acceptable cation. Suitable physiologically acceptable cations include alkali metal cations (Na⁺, K⁺), alkaline earth metal cations (Mg²⁺, Ca²⁺) or cations of organic amines, such as guanidinium morpholinium, cyclohexylammonium cation, ethylenediammonium cation, piperazonium cation (in the two latter cases, mono- or dibasic) or the cation which is derived from an amine of the formula NRₐR_{b}R_{c}, wherein Rₐ, R_{b} and R_{c} are the same or different and represent hydrogen, C₁-C₂-alkyl groups or oxyethyl groups. If it is a cation which is derived from an amine of the formula NRₐR_{b}R_{c}, it is an ammonium cation or an ammonium cation substituted with one to three C₁-C₂-alkyl groups, or an ammonium cation substituted with one to three 2-hydroxyethyl groups is preferred.

The preparation of the compounds of the formulas I and I′ can take place by known methods. The basic structure can easily be obtained by reaction of a compound of the formula RO or a functional derivative thereofwith phosphorus oxychloride and triethylamine, reaction of the product with a compound HXR₁ and acid cleavage, where R, R₁, and X have the meanings previously defined.

The preparation process for the compounds of formulas I and I′ is schematically illustrated, for example, in the following reaction equations: (the group OCH₃ in the various formulas stands representatively for the group OZ).

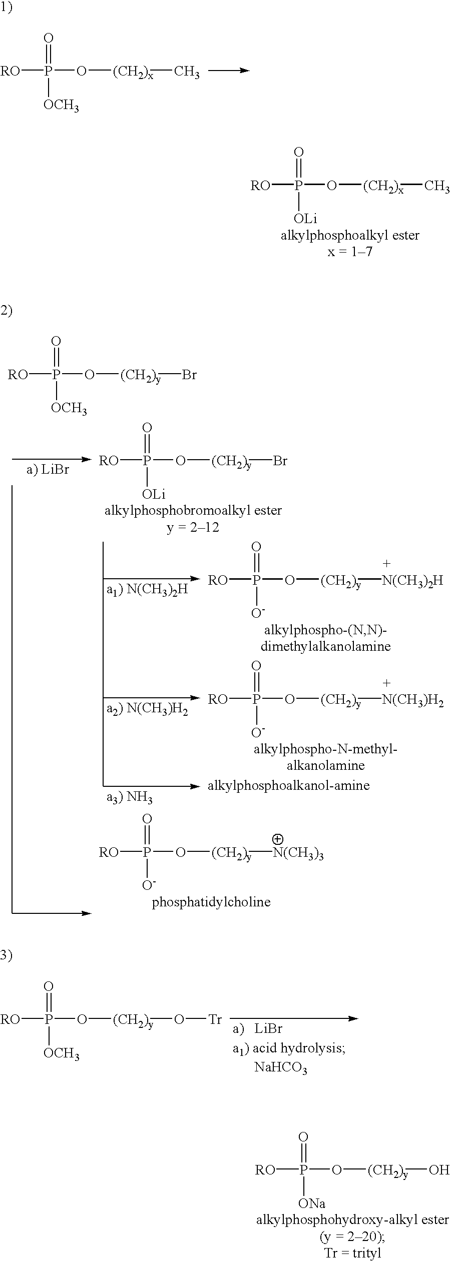

In the starting compounds of the formula III, as defined supra, hydroxyl groups, carboxyl groups, amino groups or C₁,-C₆-alkylamino groups, which occur in the radical R¹ or also in the radical R² (if X is the group NR²), can be protected by conventional protective groups. Neighboring hydroxyl groups can be protected by ketalization with an aliphatic, saturated C₃-C₆-ketone.

The protective groups are radicals which can easily be split off by hydrolysis or hydrogenation and are split off during or after the reaction. If such protective groups are not split off during the process reaction, then removal takes place after the reaction. The starting compounds frequently contain such protective groups by virtue of their preparation.

These protective groups include acyl groups which can easily be split off solvolytically, or groups which can be split off by hydrogenation. The protective groups which can be split off solvolytically can be split off, for example, by hydrolysis with dilute acids (for example, acetic acid, perchloric acid, hydrochloric acid, sulfuric acid, formic acid, trifluoroacetic acid) or by means of basic substances (potash, soda, aqueous alkali solutions, alcoholic alkali solutions, NH₃) at temperatures between −50 and 150° C., especially between 0 and 100° C. Groups which can be split off by hydrogenation, such as arylalkyl radicals (benzyl) or hydroxycarbonyl radicals (carbobenzoxy), are expediently split offby catalytic hydrogenation in the presence of conventional hydrogenation catalysts (noble metal catalysts), especially palladium catalysts or also platinum catalysts (platinum oxide), or Raney nickel, in a solvent or suspension agent, possibly under elevated pressure (for example 1-50 bar) at temperatures between 20-150° C., especially 30-100° C., preferably 40-80° C.

Suitable solvents or suspension agents for splitting off such protective groups include, but are not limited to, water, lower aliphatic alcohols, cyclic ethers, such as dioxane or tetrahydrofuran, aliphatic ethers, halogenated hydrocarbons, dimethylformamide and so forth, as well as mixtures of these agents. Suitable protective groups which can be split off by hydrogenation include benzyl, a-phenylethyl, benzyl substituted in the benzene nucleus (p-bromo- or p-nitrobenzyl radical), carbo-benzoxy, carbobenzthio and tert.butyloxycarbonyl. Examples of radicals which can be split off by hydrolysis are trifluoroacetyl, phthalyl, trityl, p-toluenesulfonyl, tert.butyloxycarbonyl, tert.butyl, dimethylmethylene and the like, as well as lower alkanoyl, such as acetyl, formyl, tert.butylcarboxy and the like.

In particular, the protective groups conventionally used in peptide syntheses and methods for their removal may be employed. In this regard, reference is made to Jesse P. Greenstein and Milton Winitz “Chemistry of Amino Acids”, New York 1961, John Wiley and Sons, Inc., Volume 2, for example page 883 et seq. The carbalkoxy group low molecular weight groups, e.g., is also suitable.

The splitting off of the group OZ (this is preferably OCH₃) may take place with alkali metal bromides or iodides, lower alkyl magnesium halides or with primary, secondary or tertiary amines, especially the corresponding lower alkylamines, such as tertiary C1-C6-alkylamines (trimethylamine). Suitable alkali metal bromides or alkali metal iodides include, but are not limited to, lithium bromide, sodium bromide, lithium iodide or sodium iodide. Suitable lower alkyl magnesium halides include, but are not limited to, methyl magnesium iodide, methyl magnesium bromide (solvents for these include lower aliphatic ethers, such as diethyl ether).

The splitting off of the group OZ from compounds of formula III takes place at temperatures between 10 and 150° C., preferably 10 and 80° C., especially 50 and 80° C.; the reaction product obtained thereby is, after removal of the solvent, dissolved in an inert agent. Suitable inert agents include saturated aliphatic C₃-C₈-ketones (ethyl methyl ketone, diethyl ketone, acetone), cyclic ethers, and non-cyclic lower aliphatic ethers (for example diethyl ether). From about 1.5 to about 3 mols of the previously mentioned cleavage agents, preferably 2 mols, are generally used per 1 mol of compound III.

The reaction of products obtained (for example compounds in which R¹ and/or R² are haloalkyl) with ammonia or with an amine of the formula NR³R⁴R⁵ takes place at temperatures between 10 and 200, preferably 20 and 150° C., especially between 40 and 80° C., with or without solvents. If a solvent or suspension agent is used, aromatic hydrocarbons, such as pentane, hexane, heptane, benzene, mesitylene, toluene or xylene; lower aliphatic ketones, such as acetone or methyl ethyl ketone; halogenated hydrocarbons, such as chloroform, trichloroethylene, carbon tetrachloride, chlorobenzene or methylene chloride; cyclic ethers, such as tetrahydrofuran or dioxane; lower aliphatic non-cyclic ethers (diethyl ether, diisopropyl ether); lower aliphatic alcohols (1-6 C-atoms), such as methanol, ethanol, isopropanol, amyl alcohol, butanol, tert.butanol; amides and N-alkyl-substituted amides of aliphatic C₁-C₄-carboxylic acids (dimethylformamide, dimethylacetamide); C₁-C₆-dialkylsufones (dimethyl sufone, tetramethyl sulfone); C₁-C₆-dialkyl sulfoxides (dimethyl sulfoxide), as well as other aprotic agents, such as N-methylpyrrolidone, tetramethylurea, hexamethylphosphoric acid triamide or acetonitrile are examples of compounds which can be used. The individual alkyl radicals of the above-mentioned solvents may contain, 1-6, or preferably, 1-4 carbon atoms. Mixtures of these agents, as well as mixtures with water, are also suitable as reaction medium. The reaction is carried out, for example, at temperatures of 0 to 200° C., preferably 20 to 150° C. or 50 to 120° C. If a solvent or dispersion agent is used, the reaction is performed at the reflux temperature of this agent.

The amination reaction is expediently carried out in the presence of basic materials, such as alkali metal hydroxides, alkali metal carbonates or tertiary amines.

The alkylation of free amino groups in the radicals R¹ and/or R² takes place at temperatures between 0 and 200° C., preferably between 20 and 150° C., especially, preferably between 20 and 80° C. This alkylation takes place, for example, by reaction with a compound of the formula R′Hal, ArS0₂0R′ and SO₂(OR′₃)₂, where Hal is a halogen atom (especially chlorine, bromine or iodine) and Ar is an aromatic radical (for example phenyl or naphthyl, optionally substituted by one or more lower alkyl radicals) and R′ is C₁-C₆-alkyl. Examples are p-toluenesulfonic acid C₁,-C₆-alkyl esters, C₁-C₆-dialkyl sulfates and C₁-C₆-alkyl halides. The alkylation reaction is optionally carried out in the presence of conventional acid-binding agents, such as alkali metal hydroxides, alkali metal carbonates, alkali metal bicarbonates, alkaline earth metal carbonates, alkali metal acetates, tertiary amines (for example trialkylamine, such as triethylamine), pyridine or also alkali metal hydrides in inert solvents or suspension agents. Suitable solvents or dispersion agents include aromatic hydrocarbons, such as benzene, toluene or xylene; aliphatic ketones, such as acetone, methyl ethyl ketones, halogenated hydrocarbons, such as chloroform, carbon tetrachloride, chlorobenzene or methylene chloride; aliphatic ethers, such as butyl ether; cyclic ethers, such as tetrahydrofuran or dioxane; sulfoxides, such as dimethyl sulfoxide; tertiary acid amides, such as dimethyl formamide, N-methylpyrrolidone or hexamethylphosphoric acid triamide; aliphatic alcohols, such as methanol, ethanol, isopropanol, amyl alcohol or tert.butanol; cycloaliphatic hydrocarbons, such as cyclohexane and the like. Aqueous mixtures of the said solvents can also be used. The reaction is advantageously performed at the reflux temperature of the particular solvent or dispersion agent. The alkylation reaction components are advantageously used in excess. The alkylation can also be carried out in the presence of tetraalkylammonium salts (especially of the halides) in combination with alkali metal hydroxides at temperatures between 0-100° C., preferably 20-80° C., in an aprotic solvent or also in chloroform or methylene chloride. Suitable aprotic solvents include tertiary amides (dimethylformamide, N-methylpyrrolidone, hexamethylphosphoric acid triamide), dimethyl sulfoxide, acetonitrile, dimethoxyethane, acetone or tetrahydrofuran.

The compounds of formula I or I′ contained in the compositions according to the present invention are, in part, novel and also an object of the invention. They exhibit outstanding cytotoxic effectiveness which has been demonstrated not only in vivo on chemically-induced mammary carcinoma of the rat, but also in vitro on leukemia cells in cell culture. Furthermore, in a clinical pilot study on female patients with mammary carcinoma, skin metastases were brought to complete healing by topical application.

The following compounds and their physiologically acceptable salts are novel.

Compounds of the formula

R-Y-PO^{⊖}₂-X-R₁, (I′)

wherein R is a saturated or unsaturated hydrocarbon radical of 12 to 24 C-atoms which may be halogen-substituted, X is oxygen, -NH- or -NR₂-, Y is oxygen or -NH-, R₁ is

a) a C₁-C₈-alkyl group, an unsaturated C₃-C₈-alkyl group or an optionally unsaturated C₃-C₈-alkyl group which is substituted by halogen, amino, C₁-C₆-alkylamino, di-C₁-C₆-alkylamino, tri C₁-C₆-alkylamino, hydroxyl, carboxyl, C₃-C₈,-cycloalkyl or phenyl, or

b) a C₂-alkyl group which is substituted by halogen, hydroxyl, carboxyl, C₃-C₈-cycloalkyl or phenyl, or

c) an unsaturated C₂-alkyl group which is substituted by di-C₁-C₆-alkylamino, tri-C₁-C₆-alkylamino, carboxyl, C₃-C₈-cycloalkyl or phenyl, or

d) a C₂-alkyl group which is substituted by amino, C₁-C₆-alkylamino, di-C₁-C₆-alkylamino or tri-C₁-C₆-alkylamino if X is oxygen, -NH- or -NR₂-, and Y is -NH-, or if X is -NR₂-, Y is oxygen, and R has the given meanings, or

e) 2-tert.butyloxycarbonylaminoethyl, 2-tert.butyloxycarbonylethyl, 2,3-isopropylidenedioxypropyl-(1), 2,3-dibenzyloxypropyl-(1), 1,3-dibenzyloxypropyl-(2) or N-C₁-C₆-alkylamino-C₂-C₆-alkyl if X is oxygen and Y and R have the given meanings, or

f) 2,3-dihydroxypropyl-(1) if X is -NH- and Y and R have the given meanings, and R₂ is 2,3-dihydroxypropyl-(1), C₁-C₈-alkyl or C₂-C₈-alkyl which is unsaturated and/or substituted with halogen, amino, C₁-C₆-alkylamino, Di-C₁-C₆-alkylamino, tri-C₁-C₆-alkylamino, hydroxyl, carboxyl, C₃-C₈-cycloalkyl or phenyl.

It has proved to be especially favorable to use the compounds of the formula I or I′ together with at least one alkylglycerol with 2 to 9 carbon atoms in the alkyl radical, which can be present in the form of an ether group attached to one of the primary or secondary OH groups of the glycerol in topical administration, as well as in other types of administration. Such alkylglycerols increase or improve the action of the compounds of formula I or I′ synergistically. Alkylglycerols with 3 to 9 C-atoms alone or in mixtures are preferred.

Therefore, a synergistically acting composition which exhibits especially favorable actions contains a) at least one compound of the formula

R-Y-PO^{⊖}₂-X-R₁

or a physiologically acceptable salt thereof, wherein R is a saturated or unsaturated hydrocarbon radical of 12 to 24 C atoms which may also be halogen-substituted, X is oxygen, -NH- or -NR₂-, and Y is oxygen or -NH-, R₁ is C₁-C₈-alkyl, C₂-C₈,-alkyl which is unsaturated and/or substituted with halogen, amino, C₁-C₆-alkylamino, di-C₁-C₆-alkylamino, tri-C₁-C₆-alkylamino, hydroxyl, carboxyl, C₃-C₈-cycloalkyl or phenyl; 2-tert.butyloxycarbonylaminoethyl, 2-tert.butyloxycarbonylethyl, 2,3-isopropylidenedioxypropyl-(1), 2,3-dibenzyloxypropyl-(1), 1,3-dibenzyloxypropyl-(2); N-C₁-C₆ alkylamino-C₂-C₆-alkyl if X is oxygen; 2,3-dihydroxypropyl-(1) if X is -NH-; and R₂- is 2,3-dihydroxypropyl-(1); C₁-C₈-alkyl; or C₂-C₈-alkyl group which is unsaturated and/or substituted with halogen, amino, C₁-C₆-alkylamino, di-C₁-C₆-alkylamino, tri-C₁-C₆-alkylamino, hydroxyl, carboxyl, C₃-C₈-cycloalkyl or phenyl; and

b) an alkylglycerol of the formula

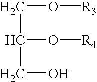

in which one of R₃ and R₄ is alkyl of 2 to 9 carbon atoms and the other is hydrogen, as well as optionally conventional inert pharmaceutical additives or diluents.

In the disclosure which follows, such a mixture is also called a cascade.

The content of a compound of the formula I or I′ in mg/ml of cascade is indicated by a subscript. For example, a cascade mixture which contains 5 mg/ml of a compound of the formula I or I′ is designated as cascade₅, and a mixture of 200 mg of a compound of the formula I or I′ per ml of cascade is designated as cascade₂₀₀.

The preparation of the alkylglycerols is known, for example, from German Offenlegungsschrift 33 43 530.

Alkylglycerol-water mixtures which contain, for example, nonylglycerol, octylglycerol, hexylglycerol, pentylglycerol, propylglycerol or ethylglycerol are preferred. Preferably, such aqueous mixtures contain 3 of the said glycerol ethers, namely, a lower (ethyl, propyl), a middle (pentyl, hexyl) and a higher (nonyl, octyl) alkyl glycerol, where the amount by weight of the lower ether is about as great as the sum of the amounts by weight of the two other glycerol ethers. The amount of water is about the same as the amount of lower glycerol ether and amounts, for example, to half of the total amount of the glycerol ethers present. Examples for such glycerol ether-water mixtures are set out in the following:

| | | glycerol | glycerol | glycerol |
|---|---|---|---|---|
| | | propyl | hexyl | nonyl |
| | water | ether | ether | ether |
| parts by | 2 | 2 | 1 | 1 |
| weight | | | | |
| | | glycerol | glycerol | glycerol |
| | | ethyl | pentyl | octyl |
| | water | ether | ether | ether |
| parts by | 2 | 2 | 1 | 1 |
| weight | | | | |

The therapeutic compositions of the present invention are especially suitable for topical administration. In order to treat skin tumors or skin metastases the composition is rubbed into the skin areas in question two to three times daily, for example with a cascade₅ to a cascade₂₀₀ composition. Harmful side effects have not been observed, even in patients who have been treated daily over a period of 3 months. The remission of the skin metastases is accompanied by a normalization of the skin, which was demonstrated by tissue sections. Several patients with skin metastases were treated in this way and complete disappearance of the mammary carcinoma skin metastases was observed.

The topical treatment with the preferred compositions of the present invention in the formulation cascade₅ to cascade₂₀₀ can also be used for the treatment of internal tumors or metastases by rubbing them into large surfaces of the skin. Therapeutically effective blood levels are thereby achieved by absorption through the skin. An advantage of this mode of administration lies in the fact that the compositions cascade₅ to cascade₂₀₀ are tolerated by the skin without problems.

This preferred type of composition of the medicament according to the invention in the form of cascade₅ to cascade₂₀₀ is also well suited for the preparation of suppositories for rectal insertion. Internal tumors and internal metastases can also be effectively treated therewith.

Another route of administration of the composition of this invention is instillation into pre-formed body cavities. This type of administration is especially suitable for pleural carcinoses, malignant ascites, malignant pericardial discharges and bladder carcinomas. In this case, the anti-tumor compounds of the formula I are used either alone or in combination with conventional inert carriers or diluents, especially also with cascades.

For systemic administration, oral or intravenous administration is effective.

For oral administration, the compounds of formula I are advantageously used in the form of a potable solution. Suitable potable carriers for this purpose include milks, cocoa, fruit juices and drinking water. The preparation of such potable solutions can involve, for example, diluting a concentrated alcoholic solution of a compound of the formula I or I′ with water or with another one of the previously mentioned carriers. In the case of rats, daily doses of 20, 40 and 60 mg/kg body weight of oleylphosphocholine, lead to a complete remission of chemically-induced mammary carcinomas. These compounds proved to be more effective and more compatible than 1-octadecyl-2-methyl-rac-glycero-3-phosphocholine. The tumor model used for these experiments was a so-called hard model. This means that the findings obtained with this model can also be applied to humans.

For intravenous administration via intravenous infusion therapy, the compounds of the invention are preferably used in physiological salt solution. Other infusion solutions may also be used. Dosage in humans for such solutions is, for example, 1-10 mg/kg body weight.

Finally, several types of administration of the compositions according to the invention can be used in combination, where their special topical compatibility means that application to the skin, such as rubbing, is combined with another form of administration.

A carrier mixture for the compounds of the formula I or I′, which has proved to be especially useful, consists of a mixture of about 4 parts by weight of propylglycerol and 2 parts by weight of hexylglycerol and nonylglycerol each.

The topical use of the composition according to the invention in the especially preferred form of cascade₅ to cascade₂₀₀ over a period of time of several months showed that local toxicity was limited to an increased desquamation of the skin, similar to the localized use of acetylsalicyclic acid.

Thus, the invention makes available novel therapeutic compositions for the treatment of tumors and provides, not only a further anti-tumor agent but provides, for the first time, an agent which, as has been proven in clinical experiments, is also effective in the case of topical use. New possibilities are thereby opened up for the treatment of tumor patients.

For the preparation of therapeutic compositions, at least one compound of the formula I or I′ is combined with conventional pharmaceutical carrier materials, diluents or other adjuvant materials to form pharmaceutical compositions in therapeutically usable form. For example, a compound of the formula I or I′ , or a physiologically acceptable salt thereof, is admixed or homogenized with conventional carriers, diluents or adjuvants at temperatures between 20 and 120° C., preferably between 30 and 100° C., the mixture obtained thereby is processed into dosage unit compositions containing 5 to 2000 mg, preferably 10 to 500 mg, especially 30 to 400 mg of the active ingredient of the formula I or I′ per dose, and the composition is filled into hollow cells or capsules of appropriate size, or the composition if granulated and, after optionally adding further adjuvants, the granulate is compressed into tablets.

For example, a compound of the formula I or I′ is admixed with starch, cellulose, lactose, formalin-casein, modified starch, magnesium stearate, calcium hydrogen phosphates, highly dispersed silicic acid, talcum, phenoxyethanol or mixtures of two or more of these inert carriers, the resulting mixture is granulated, optionally with an aqueous solution of gelatin, starch, polyvinylpyrrolidone, vinylpyrrolidone-vinyl acetate copolymer, polyoxyethylsorbitol monooleate or a mixture of these, the granulate is homogenized optionally with one or more of the above mentioned adjuvants, and the homogenized granulate is compressed into tablets or is filled into capsules, each containing a dosage unit of 5 to 2000 mg of active ingredient of the formula I or I′. Alternatively, a compound of the formula I or I′ or a physiologically acceptable salt thereof is suspended or homogenized in molten hard fat after the addition of soybean lecithin optionally with 0.1 to 0.5 parts by weight of phenoxyethanol, based on 1 part by weight of compound I or I′, at a temperature between 33 and 37° C., and the mixture is poured into hollow cells of suitable size to form dosage units containing 5 to 2000 mg of active ingredient, optionally with 0.1 to 0.5 parts by weight of phenoxyethanol, based on 1 part by weight of compound I or I′. A compound of the formula I or I′ or a physiologically acceptable salt thereof may also be homogenized at a temperature between 50 and 120° C., preferably between 50 and 100° C., optionally in the presence of one or more emulsifiers or 0.1 to 0.5 parts by weight of phenoxyethanol, based on 1 part by weight of compound I or I′, with paraffin, petroleum jelly, aliphatic alcohols of 12 to 25 carbon atoms, aliphatic monocarboxylic acids of 15 to 20 carbon atoms, sorbitol monopalmitate, polyoxyethylene-polyol fatty acid esters or a mixture of these, the mixture thus obtained is emulsified between 50 and 120° C. with water, optionally after the addition of apolyhydroxy lower aliphatic alcohol or phenoxyethanol. Finally, a compound of the formula I or I′ or a physiologically acceptable salt thereof may be dissolved in water or vegetable oil, optionally in the presence of 0.1 to 0.5 parts by weight of phenoxyethanol, based on 1 part by weight of compound I or I′, optionally in the presence of an emulsifier, at a temperature between 30 and 100° C., and the solution thus obtained may be diluted with an amount of water or vegetable oil so that the final solution contains 0.05 to 10% by weight, preferably 0.1 to 5% by weight of active ingredient of the formula I or I′.

Suitable emulsifiers include non-ionic emulsifiers as well as ionic emulsifiers. Examples of non-ionic emulsifiers are triglyceride mixtures of saturated vegetable fatty acids with 8, 10 and 12 carbon atoms, or emulsifiers based on polyaddition products of ethylene oxide, such as alkyl- and acyl-substituted polyaddition products of ethylene oxide, polyethylene glycol fatty acid esters, reaction products of ethylene oxide with castor oil or hydrogenated castor oil, ester of hydrogenated castor oil fatty acids with oxyethylated glycerol. The non-ionic emulsifiers may also be based on fatty acid amides or fatty acid condensation products with hydrophilic groups. Examples of ionic emulsifiers are emulsifiers based on fatty acid monoesters of glycerol or other polyhydroxy alcohols (lunacera alba).

If, in the preparation of the therapeutic compositions described above, the active ingredient of formula I or I′ is used together with a glycerol ether of the formula II or a mixture of such glycerol ethers, synergistic enhancement of the anti-tumor action is observed.

For this purpose, the active ingredients of formula I or I′ are used with 1 to 30 parts by weight, preferably 2 to 20 parts by weight, based on 1 part by weight of compound I or I′, of at least one glycerol ether of the formula II or a mixture of such glycerol ethers, as well as optionally 0.5 to 30 parts by weight, preferably 1 to 20 parts by weight of water, also based on 1 part by weight of compound I or I′. The glycerol ethers may be admixed with the compound of compounds of the formula I or I′ at the beginning or also in a later stage of preparation of the therapeutic compositions according to the present invention.

The compounds of the formulas I and I′ exhibit very effective anti-tumor action against 7,12-dimethylbenzanthracene-induced mammary cancer of the rat, as well as against methylnitrosourea-induced mammary carcinoma of the rat.

For example, a dose of 10 mg/kg body weight of rat results in cessation of growth of the tumors, and higher doses cause complete disappearance of the growth.

The lowest already active dose is 5 mg/kg when administered orally and 5 mg/kg intravenously. The general effective dose range in the animal model described above is 5 to 50 mg/kg, preferably 15 to 32 mg/kg both orally and intravenously.

The direction of action of the compounds according to the invention is comparable with the action of the known medicament tamoxifen, but the action is stronger and of longer duration than that of tamoxifen.

Indications for the compounds according to the invention are mammary cancer and other human types of cancer.

The pharmaceutical compositions contain, in general, between 5-2000 mg, for example 10-400 mg of the compounds according to the invention.

The administration can take place, for example, in the form of tablets, capsules, pills, dragees, cones, salves, gels, creams, powders, dusting powders, aerosols or in liquid form. Examples of liquid forms of administration include oily, alcoholic or aqueous solutions, as well as suspensions and emulsions. Preferred forms of administration are tablets which contain between 40 and 400 mg or solutions which contain between 0.1 and 5% of active substance.

The individual dose of the active components according to the invention can be, for example

a) in the case of oral forms, between 5-100 mg/kg body weight, preferably 15-50 mg/kg body weight,

b) in the case of parenteral forms (including intravenous intramuscular compositions) between 5-100 mg/kg body weight,

c) in the case of forms suitable for local administration to the skin and mucous membranes (for example in the form of solutions, lotions, emulsions, ointments and so forth) between 50-2000 mg, preferably 80-1500 mg.

(The doses are, in each case, referred to the free base).

For example, 1 tablet with a content of 40-400 mg of active substance can be administered 3 times daily or, for example, in the case of intravenous injection, one ampule of 1-5 ml containing 50-250 mg of substance can be administered 1-5 times per day. In the case of oral administration, the minimum daily dose is, for example, 120 mg; the maximum daily dose in the case of oral administration should not be more than 100 mg/kg body weight.

The acute toxicity of the compounds according to the invention on the mouse (expressed by the LD 50 mg/kg; method according to Miller and Tainter; Proc. Soc. Exper. Biol. a. Med. 57 (1944) 261)) is for example, in the case of oral administration, between 200 and 450 mg/kg body weight.

In addition to their efficiency as anticancer agents, the compounds and compositions of the invention are useful as antiviral agents. Hexadeclyphosphocholine can be used as an antiviral agent as well. These compounds may be used as is, or in combination with alkylglycerols, in the manner and form described supra in treating cancer.

The compounds and compositions are particularly useful in the treatment of conditions involving viruses with a lipid membrane. Exemplary of such viruses are wart (Verruca) viruses, such as Verruca accuminata, Verruca plantaris, Verruca senilis, and Verruca vulgaris, as well as Epidermodysplasia Verruciformis, influenza virus, hepatitis C virus, adenovirus, and Herpes simplex virus. In the treatment of conditions involving viruses such as those described supra, the mode of administration will vary; however, it is preferred to administer the compounds and compositions topically, although other forms of administration, such as those set forth supra, are also appropriate.

The compositions can be used in human medicine, veterinary medicine, as well as in agriculture, alone or in admixture with other pharmacologically active materials.

The following examples illustrate the present invention and will enable others skilled in the art to understand it more completely. It should be understood, however, that the invention is not limited solely to the particular examples given below.

### EXAMPLE 1

Hexadecylphosphoethanolamine

(phosphorylation, ring closure and ring opening)

Hexadecanol (1 mol, 243 g) and triethylamine (1.8 mol, 180 g) are dissolved in 1.5 liters of THF (tetrahydrofuran) and the solution is added dropwise to a vigorously stirred solution of phosphorus oxychloride (1.2 mol, 184 g) in 120 ml of THF in such a manner that the temperature in the reaction vessel (three-necked, 5 liters, with dropping funnel, thermometer and stirrer) does not exceed 10° C. For the acceleration of the procedure, the reaction vessel is cooled with an ice-salt mixture. Immediately after the dropwise addition, the reaction is concluded (detection via TLC in ether: Rf values of 0.8 for the starting product, of 0.0 for the reaction product after hydrolysis with water).

One removes the ice bath and drops into the reaction mixture, with vigorous stirring, a solution of ethanolamine (1.5 mols, 92 g) and triethylamine (1.8 mols, 180 g) in 1 liter of dioxane in such a manner that the temperature in the reaction vessel increases to 65 to 70° C. The ring formation is then concluded (detection by TLC in ether: Rf value of 0.2). One filters off from precipitated triethylamine hydrochloride while still warm and mixes the filtrate at 40 to 50° C. with 1.5 liters of 2N formic acid. After 15 minutes, the ring opening is concluded (detection by TLC in ether: Rf value 0.0; TLC in chloroform/methanol/acetic acid/water 100:60:20:5 in vol.; Rf value 0.8). One cools to −20° C. and filters off the precipitate which consists substantially of pure hexadecylphosphoethanolamine. In the case of slight impurities, a chromatographic purification follows (see Example 2). Microanalysis (M.W. 365.50):

| | | | | | | | | |
|---|---|---|---|---|---|---|---|---|
| calc. (%): | C | 59.15 | H | 11.03 | N | 3.83 | P | 8.48 |
| found (%): | | 59.01 | | 10.95 | | 3.79 | | 8.31 |

### EXAMPLE 2

2-Hexadecylphosphoethanolamine

(phosphorylation, ring closure, ring opening)

The reaction takes place as described in Example 1 but for 0.1 mol. In order to achieve good yields, the phosphorylation conditions must be somewhat modified, i.e., the temperature in the phosphorylation step is increased to 25° C. Otherwise one proceeds and works up as described. Microanalysis (M.W. 365.50)

| | | | | | | | | |
|---|---|---|---|---|---|---|---|---|
| calc. (%) | C | 59.15 | H | 11.03 | N | 3.83 | P | 8.48 |
| found (%) | | 58.96 | | 10.91 | | 3.69 | | 8.39 |

### EXAMPLE 3

Oleylphosphomethyl ester, sodium salt+1 H₂O

(phosphorylation, methanolysis and LiBr cleavage)

The phosphorylation step takes place as in Example 1. For the methanolysis, the reaction mixture is mixed with methanol (10 mols, 320 g) and triethanolamine (1.8 mols, 180 g) at 20° C. The methanolysis is concluded after 30 minutes. One mixes with 1.5 liters of hexane and 1.5 liters of water, shakes thoroughly and removes the solvent from the hexane phase. The oily residue is boiled under reflux with LiBr (2 mols, 174 g) in 1.5 liters of ethyl methyl ketone. After one hour, the reaction is complete. One removes the solvent, takes up in a mixture of 1 liter of methanol/water/chloroform, shakes thoroughly and isolates the lower chloroform phase which contains the product. For the conversion into the sodium salt, one treats the chloroform phase with 1 liter of saturated NaCl solution. The chloroform phase is isolated and evaporated. One purifies the product by chromatography on silica gel. Microanalysis (M.W. 402.50)

| | | | | | | |
|---|---|---|---|---|---|---|
| calc. (%): | C | 56.70 | H | 10.02 | P | 7.70 |
| found (%): | | 56.65 | | 9.98 | | 7.45 |

The allyl ester was prepared correspondingly, as were the methyl- and allylphosphoric acid esters with tetradecanol, hexadecanol, octadecanol and eicosanol.

### EXAMPLE 4

Hexadecylphosphohexyl ester, sodium salt+1 H₂O

(phosphorylation with phosphorus oxychloride, phosphorylation with hexadecylphosphoric acid dichloride, methanolysis, cleavage with LiBr)

The phosphorylation of hexadecanol takes place as described in Example 1. The reaction mixture is further reacted directly with dropwise addition of hexanol (1.5 mols, 303 g) and triethanolamine (1.8 mols, 180 g) in 1.5 liters of THF. The temperature is now increased to 30° C. After two hours, the reaction has finished.

Methanolysis as well as the LiBr cleavage take place as described in Example 5. Microanalysis (M.W. 446.59)

| | | | | | | |
|---|---|---|---|---|---|---|
| calc.(%): | C | 59.17 | H | 10.83 | P | 6.94 |
| found (%): | | 59.08 | | 10.74 | | 6.71 |

The following alkyl esters were prepared analogously: hexadecylphosphobutyl, -octyl, -decyl and -dodecyl ester.

### EXAMPLE 5

Hexadecyphosphoglycol ester sodium salt+1 H₂O

(phosphorylation, ring closure with glycol, ring opening)

The phosphorylation takes place as described in Example 1. The reaction mixture is further reacted directly with the dropwise addition of ethylene glycol (1.5 mols, 93 g) and triethanolamine (1.8 mols, 180 g) in 1.5 liters of THF. For the completion of the ring formation, the temperature is increased to 60° C. After 2 hours at this temperature, the reaction is concluded. One filters off the precipitated triethanolamine hydrochloride over a porcelain frit and mixes the filtrate, while vigorously stirring at 20° C., with 1.5 liters of water. After 2 hours, the hydrolysis has finished. One removes the solvent from the upper THF phase by evaporation in a vacuum. The residue is mixed with a mixture of chloroform/methanol/half saturated NaCl solution, shaken, and phase separation is awaited. The lower chloroform phase contains the product. One removes the solvent and purifies the product by chromatography (Example 2). Microanalysis (M.W. 406.48)

| | | | | | | |
|---|---|---|---|---|---|---|
| calc. (%): | C | 53.19 | H | 9.92 | P | 7.62 |
| found (%): | | 53.07 | | 9.73 | | 7.53 |

The following glycol esters were prepared analogously: tetradecylphosphoglycol ester, octadecylphosphoglycol ester, oleylphosphoglycol ester.

### EXAMPLE 6

Hexadecylphospho hydroxyehylamide, sodium salt+1 H₂O

(phosphorylation, ring closure with ethanolamine, opening with potassium carbonate in water)

The phosphorylation takes place as described in Example 1, as does the ring closure. After removal of the triethylamine hydrochloride, the filtrate is mixed, while vigorously stirring, with 1 liter 1 M potassium carbonate solution in water. After 1 hour, the ring opening is finished. One removes the solvent in the upper THF phase, takes up in a mixture of 1 liter of chloroform/methanol/half saturated NaCl solution, shakes well and separates the chloroform phase. After removal of the solvent, the product is chromatographed and purified on silica gel. Microanalysis (M.W. 405.50)

| | | | | | | | | |
|---|---|---|---|---|---|---|---|---|
| calc. (%): | C | 53.32 | H | 10.19 | N | 3.46 | P | 7.64 |
| found (%): | | 53.26 | | 10.07 | | 3.21 | | 7.59 |

The following compounds were prepared analogously: tetradecyl-, octadecyl-, oleylphospho- hydroxyethylamide.

### EXAMPLE 7

Hexadecylphosphoglycerol, sodium salt+1 H₂O

(phosphorylation with phosphorus oxychloride, phosphorylation with the phosphoric acid dichloride formed thereby, methanolysis, LiBr cleavage, hydrolysis in 70% acetic acid)

The phosphorylation corresponds to Example 1. The reaction mixture is further reacted directly with dropwise addition of 1,2-isopropylidene-glycerol (1.5 mols, 198 g) and triethylamine (1.8 mols, 180 g) in 1.5 liters of THF. After the dropwise addition, the temperature is increased to 30° C. The reaction is finished after two hours. Methanolysis takes place according to Example 5, as does the LiBr cleavage. The reaction product, sodium salt, is taken up in 2 liters of 70% acetic acid and heated to 60° C. The resultant acetone is removed in a slight vacuum (waterpump vacuum). The reaction is concluded after 2 hours. One mixes with 2 liters of water and extracts with 2 liters of chloroform. The chloroformphase is treatedwith 2 liters of0.5 M sodium carbonate solution and, after phase separation, separated. One removes the solvent and chromatographs on silica gel. Microanalysis (M.W. 436.51)

| | | | | | | |
|---|---|---|---|---|---|---|
| calc. (%): | C | 52.28 | H | 9.70 | P | 7.10 |
| found (%): | | 52.13 | | 9.59 | | 6.91 |

The following glycerol esters were prepared analogously: tetradecyl-, octadecyl-, oleylphosphoglycerol.

### EXAMPLE 8

Hexadecylphosphoric acid (N,N)-bis-(chloroethyl)-amine Na salt+H₂O (phosphorylation with phosphorus oxychloride, amide formation with bis-(chloroethyl) -amine, hydrolysis)

The phosphorylation step corresponds to Example 1. The reaction mixture is further reacted directly with the dropwise addition of bis-(chloroethyl)-amine in 1.0 liters of THF. Thereafter, triethylamine (0.4 mols, 40 g) in 0.5 liters of THF is added thereto. After 3 hours at 20° C., the reaction is finished. One separates the precipitated triethylamine hydrochloride over a porcelain frit and mixes the filtrate, while vigorously stirring, with 1 liter 1 M acetic acid for hydrolysis. After 4 hours, the upper THF phase is separated, freed from solvent and taken up in 1 liter of chloroform/methanol/0.5 M sodium carbonate. The chloroform phase is removed, the solvent is removed therefrom, and the product is purified by chromatography on silica gel. Microanalysis (M.W. 486.452)

| | | | | | | | | | | |
|---|---|---|---|---|---|---|---|---|---|---|
| calc. (%): | C | 49.38 | H | 8.91 | C1 | 14.58 | N | 2.88 | P | 6.37 |
| found (%): | | 49.21 | | 8.75 | | 14.11 | | 2.76 | | 6.31 |

The following compounds were prepared analogously: tetradecyl-, octadecyl-, oleylphosphoric acid-(N,N)-bis-(chloroethyl)-amide.

### EXAMPLES OF PHARMACEUTICAL COMPOSITIONS

Example of a solution:

25 g of 1-n-propyloxy-2,3-propanediol, 12.5 g of 1-n-hexyloxy-2,3-propanediol, 12.5 g of 1-n-nonyloxy-2,3-propanediol, 44 g of water and 1 g of phenoxyethanol are mixed and 5 g of hexadecylphosphoglycerol are dissolved in this mixture. The solution is freed from visible particles by filtration over suitable filters.

1 g of solution contains 50 mg of hexadecylphosphoglycerol. Example of an ointment:

5 g of hexadecylphosphoglycerol are suspended in 35 g of very viscous paraffin, 30 g of emulsifying cetylstearyl alcohol and 30 g of white vaseline are added thereto and melted. The melt is stirred until cool. A homogeneous active material distribution is achieved by working up of the cooled melt by means of a suitable homogenizing apparatus (for example a three-roll mill). 1 g of the hydrophilic salt contains 50 mg of hexadecylphosphoglycerol.

Example of an emulsion:

11.83 g of 1-n-propyloxy-2,3-propanediol, 5.91 g of 1-n-hexyloxy-2,3-propanediol, 5.91 g of 1-n-nonyloxy-2,3-propanediol, 20.35 g of water and 1.0 g of phenoxyethanol are mixed and 5 g of hexadecylphosphoglycerol dissolved in this mixture. 30 g of white vaseline, 15 g of cetyl alcohol and 5 g of sorbitol monopalmitate are melted on a waterbath, heated to 70° C. and the active material solution, also heated to 70° C. is emulsified in the fat phase with the aid of a high-speed dispersing apparatus. Subsequently, the cream is cooled to 30° C., while stirring.

1 g of water-in-oil emulsion contains 50 mg of hexadecylphosphoglycerol.

Example of capsules:

1.25 kg of hexadecylphosphoglycerol are dissolved in 5 kg of chloroform and 1.25 kg of Aerosil are suspended in this solution. Subsequently, the solvent is removed in a vacuum. The dry mass is passed though a 1 mm sieve and once again dried in a vacuum at 30° C. in order to remove the last residues of solvent. The resulting granulate is filled in known manner with a suitable capsule machine into hard gelatin capsules, size 00, in an amount of 500 mg. One capsule contains 250 mg of hexadecylphosphoglycerol.

Example of a lyophilisate:

500 g of mannitol are dissolved in 3 liters of water for injection purposes in an atmosphere of nitrogen, 50 g of hexadecylphosphoglycerol are dispersed in the solution with the aid of a high-speed homogenization apparatus, and the dispersion is diluted to 4 liters with water for injection purposes. This milky dispersion is converted into a slightly opalescing colloid-disperse system by ultrasonic treatment or with the aid of a slot homogenizer.

Under aseptic conditions, the composition is now sterile filtered over a membrane filter of 0.22 μm pore width and filled in 40 ml amounts into 100 ml injection bottles in a nitrogen atmosphere. One provides the bottles with freeze-drying stoppers and lyophilizes in a suitable plant. After drying, the lyophilizate is gassed with sterile, dry nitrogen, and the bottles closed in the plant. The stoppers are secured with a flanged cap.

For intravenous use, the lyophilizate is reconstituted in 100 ml of water for injection purposes. One bottle contains 500 mg of hexadecylphosphoglycerol.

The following example demonstrates the efficacy of compounds in accordance with the invention in the treatment of virus mediated disorders.

### EXAMPLE 9

A 37 year old male presented a wart-shaped excrescence of increasing size (diameter: about 5 mm; height: about 3 mm), formed at the site of a stab wound on his right middle finger after an initial inflammatory reaction. The excrescence grew over a period of about 1.5 years. There was heavy vascularization at the site, as well as superficial furrowing. The position of the wart (side facing the ring finger), made it quite susceptible to injury.

The patient was treated by topically administering a composition containing 40 mg/ml hexadecylphosphocholine, 317 mg/ml of 3-propoxypropyleneglycol, 158 mg/ml of hexyloxypropyleneglycol, and 158 mg/ml of nonyloxypropyleneglycol, in solution, and then a solution containing 60 mg/ml of the hexadecy/phosphocholine, and the other components above, in the same amounts. The solutions were administered topically, 2-3 times per day.

The excrescence rapidly developed a horny surface, and regressed. Cornified residues were removable without bleeding. The treatment was terminated before the surrounding skin level was reached. This took about 6 weeks. When the patient was examined about 8 weeks later, the condition was stable. There was a slight reddening of the skin, which disappeared after about 2 weeks. It is believed that this was the result of regeneration of the epidermis in the excrescence region. There was a slight peeling of healthy skin around the excrescence which, inevitably, received treatment as well, and which was defacto discontinued after treatment of the excrescence ended. There was no apparent damage to the surrounding, healthy skin regions.

While the present invention has been illustrated with the aid of certain specific embodiments thereof, it will be readily apparent to others skilled in the art that the invention is not limited to these particular embodiments, and that various changes and modifications may be made without departing from the spirit of the invention or the scope of the appended claims.

I Claim

1. A compound of the formula
R-Y-P^{⊖}₂-X-R₁ (I′)
wherein R is a saturated or unsaturated hydrocarbon radical of 12 to 24 carbon atoms which may also be halogen-substituted,
X is oxygen, -NH or -NR₂-,
Y is oxygen or -NH-,
R₁ is C₁-C₈-alkyl, unsaturated C₃-C₈-alkyl, optionally unsaturated C₃-C₈-alkyl which is substituted with halogen, amino, C₁-C₆-alkylamino, di-C₁-C₆-alkylamino, tri-C₁-C₆-alkylamino, hydroxyl, carboxyl, C₃-C₈-cycloalkyl or phenyl, C₂-alkyl which is substituted with hydrogen, hydroxyl, carboxyl, C₃-C₈-cycloalkyl or phenyl; unsaturated C₂-alkyl which is substituted with di-C₁-C₆-alkylamino, tri-C₁-C₆-alkylamino, C₃-C₈-cycloalkyl orphenyl; C₂-alkyl which is substituted with amino, C₁-C₆-alkylamino, di-C₁-C₆-alkylamino or tri-C₁-C₆-alkylamino when X is oxygen, -NH- or -NR₂- and Y is -NH-, or when X is -NH- or -NR₂-, Y is oxygen and R has the meanings previously defined; 2-tert. -butoxycarbonyl-aminoethyl, 2-tert.-butoxycarbonylethyl, 2,3-isopropylidenedioxy-propyl-(1), 2,3-dibenzyloxypropyl-(1), 1,3-disbenzyloxypropyl-(2) or N-C₁-C₆-alkylamino-C₂-C₆-alkyl when X is oxygen and Y and R have the meanings previously defined; 2,3-dihydroxypropyl-(1) when X is -NH- and Y and R have the meanings previously defined; and
R₂ is 2,3-dihydroxypropyl-(1), C₁-C₈-alkyl or C₂-C₈-alkyl which is unsaturated and/or substituted with halogen, amino, C₁-C₆-alkylamino, di-C₁-C₆-alkylamino, tri-C₁-C₆-alkylamino, hydroxyl, carboxyl, C₃-C₈-cycloalkyl or phenyl;
and physiologically acceptable salts thereof.

2. The compound of claim 1, wherein R is 16-20 carbon atoms long, X and Y are oxygen, and R₁, is CH₂CH₂NR2R3R4, wherein each of R2, R3 & R4 are alkyl.

3. The compound of claim 2, wherein R is 17-19 carbon atoms long.

4. The compound of claim 2, wherein R2, R3 & R₄ are methyl, ethyl, propyl, 2-hydroxyethyl, cyclopropyl, cyclobutyl, or cyclopentyl.

5. The compound of claim 2, wherein R2 & R3 are methyl, and R₄ is methyl or ethyl.

6. The compound of claim 5, wherein R is heptadecyl, octadecyl, nonadecyl, eicosyl, cis-heptadec-8-enyl, cis-octadec-9-enyl, cis-nonadec-10-enyl, or cis-eicos-11-enyl.

7. The compound of claim 2, wherein R contains at least one double or triple bond.

8. The compound of claim 2, wherein R comprises at least one double bond, in syn position.

9. The compound of claim 8, wherein said at least one double bond is at a position between C6 and C(ω-3).

10. The compound of claim 8, wherein said at least one double bond is at the terminus of R.

11. The compound of claim 1, wherein R comprises two double bonds.

12. The compound of claim 11, wherein said two double bonds are cis double bonds.

13. The compound of claim 12, wherein said two double bonds are located between C6 and C(ω-6), and C10 and C(ω-3).

14. The Compound of claim 12, wherein said two double bonds are located between C6 and C(ω-6), and at the terminus of R.

15. Composition comprising the compound of claim 1, and an inert, physiologically compatible carrier.

16. The composition of claim 15, further comprising human serum albumin.

17. The composition of claim 16, wherein said compound and human serum albumin are present in a ratio of from about 1:1 to 3:1.

18. The composition of claim 15, further comprising cholesterol.

19. The composition of claim 18, wherein said compound and said cholesterol are present in a ratio of from about 1:1 to about 1:1.2.

20. The composition of claim 17, wherein said compound and said human serum albumin are present in a ratio of from about 1:1 to about 2:1.

21. Composition comprising the compound of claim 1, and at least one alkylglycerol of formula 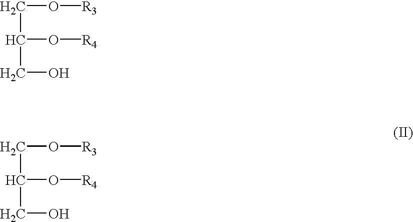
wherein one of R₃ & R₄ is C2-C9 alkyl, and the other is hydrogen.

22. The composition of claim 21, comprising propoxypropyleneglycol, hexyloxypropyleneglycol, and nonyloxypropyleneglycol.

23. Composition comprising
a) a compound of the formula
R-Y-PO^{⊖}₂-X-R₁
wherein R is a saturated or unsaturated hydrocarbon radical of 12 to 24 carbon atoms, which may also be halogen-substituted;
X is oxygen, -NH- or -NR₂-;
Y is oxygen or -NH-;
R₁ is C₁-C₈-alkyl, C₂-C₈-alkyl which is unsaturated or optionally substituted with halogen, amino, C₁-C₆-alkylamino, di-C₁-C₆-alkylamino, tri-C₁-C₆-alkylamino, hydroxyl, carboxyl, C₃-C₈-cycloalkyl, phenyl, 2-tert.-butoxycarbonylaminoethyl, 2-tert.-butoxycarbonylethyl, 2,3-iso-propylidenedioxypropyl-(1), 2,3-dibenzyloxypropyl-(1), 1,3-dibenzyloxypropyl-(2), N-C₁-C₆-alkylamino-C₂-C₆-alkyl when X is oxygen, or 2,3-dihydroxypropyl-(1) when X is -NH-, and
R₂ is 2,3-dihydroxypropyl-(1), C₁-C₈-alkyl or C₂-C₈-alkyl which is unsaturated or optionally substituted with halogen, amino, C₁-C₆-alkylamino, di-C₁-C₆-alkylamino, tri-C₁-C₆-alkylamino, hydroxyl, carboxyl, C₃-C₈-cycloalkyl or phenyl, provided, however, that hexadecylphosphocholine is excepted, or a physiologically acceptable salt thereof; and
b) an alkylglycerol of the formula 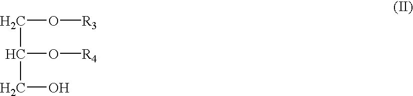
wherein one of R₃ and R₄ is alkyl of 2 to 9 carbon atoms and the other is hydrogen.

24. The composition of claim 23, wherein
R is alkyl or alkenyl of 14 to 20 carbon atoms,
X is oxygen, and
R₁ is trialkylammoniumethyl of 1 to 3 carbon atoms per alkyl group.

25. The composition of claim 23, which contains 5 to 200 mg of a compound of the formula I per ml of alkylglycerol.

26. The composition of claim 23, which contains as component b) a mixture of three alkylglycerols, one of which is nonyl- or octylglycerol, another is hexyl- or pentylglycerol, and the third is propyl- or ethylglycerol, and water.

27. The composition of claim 23, in potable form and comprising from 5 to 100 mg of said composition.

28. The composition of claim 23, in a physiological saline solution.

29. A method for treating a patient suffering from cancer, comprising administering to said patient an anticancer effective amount of the compound of claim 1.

30. The method of claim 29, comprising administering said compound topically.

31. The method of claim 29, comprising administering said compound intravenously.

32. The method of claim 29, comprising administering said compound orally.

33. The method of claim 29, wherein said patient suffers from mammary cancer.

34. The method of claim 29, wherein said patient suffers from skin cancer.

35. A method for treating a patient suffering from cancer, comprising administering to said patient an amount of the composition of claim 16 sufficient to alleviate said cancer.

36. A method for treating a patient suffering from cancer, comprising administering to said patient an amount of the composition of claim 8 sufficient to alleviate said cancer.

37. The method of claim 35, wherein said cancer is mammary cancer or skin cancer.

38. The method of claim 35, comprising administering said composition topically, intravenously, or orally.

39. The method of claim 36, wherein said cancer is mammary cancer or skin cancer.

40. The method of claim 36, comprising administering said composition topically, intravenously, or orally.

41. A method for treating a patient suffering from cancer, comprising administering a therapeutically effective amount of the composition of claim 21 to said patient.

42. The method of claim 41, wherein said cancer is mammary cancer or skin cancer.

43. The method of claim 41, comprising administering said composition topically, intravenously, or orally.

44. A method for treating a patient suffering from cancer, comprising administering to said patient an anticancer effective amount of the composition of claim 23.

45. The method of claim 44, wherein said cancer is mammary cancer or skin cancer.

46. The method of claim 44, comprising administering said composition topically, intravenously, or orally.

47. A method for treating a patient with a viral infection comprising administering an antiviral effective amount of the compound of claim 1 to said patient.

48. The method of claim 47, comprising administering said compound topically, intravenously, or orally.

49. The method of claim 47, wherein said viral infection is caused by a virus with a lipid membrane.

50. The method of claim 47, wherein said viral infection is caused by Verruca accuminata, Verruca plantaris, Verruca senilis, Verruca vulgaris, or Epidermodyplasia verruciformis.

51. The method of claim 47, wherein said viral infection is caused by influenza virus, hepatitis C virus, adenovirus, human immunodeficiency virus or herpes simplex virus.

52. A method for treating a patient with a viral infection, comprising administering an antiviral effective amount of the composition of claim 15 to said patient.

53. The method of claim 52, wherein said composition comprises human serum albumin or cholesterol.

54. The method of claim 52, comprising administering said composition topically, intravenously, or orally.

55. The method of claim 52, wherein said viral infection is caused by a virus with a lipid membrane.

56. The method of claim 52, wherein said viral infection is caused by Verruca accuminata, Verruca plantaris, Verruca senilis, Verruca vulgaris, or Epidermodyplasia verruciformis.

57. The method of claim 52, wherein said viral infection is caused by influenza virus, hepatitis C virus, adenovirus, human immunodeficiency virus, or herpes simplex virus.

58. A method for treating a patient with a viral infection, comprising administering an antiviral effective amount of the composition of claim 21 to said patient.

59. The method of claim 58, comprising administering said composition topically, intravenously or orally.

60. The method of claim 58, wherein said viral infection is caused by a virus with a lipid membrane.

61. The method of claim 58, wherein said viral infection is caused by Verruca accuminata, Verruca plantaris, Verruca senilis, Verruca vulgaris or Epidermodyplasia Verrucifornis.

62. The method of claim 58, wherein said viral infection is caused by influenza virus, hepatitis C virus, adenovirus, human immunodeficiency virus, or herpes simplex virus.

63. A method for treating a patient with a viral infection, comprising administering an antiviral effective amount of the composition of claim 23 to said patient.

64. The method of claim 63, comprising administering said composition topically, intravenously, or orally.

65. The method of claim 63, wherein said viral infection is caused by a lipid membrane containing virus.

66. The method of claim 63, wherein said viral infection is caused by Verruca accuminata, Verruca plantaris, Verruca senilis, Verruca vulgaris, or Epidernodyplasia verruciformis.

67. The method of claim 63, wherein said viral infection is caused by influenza virus, hepatitis C virus, adenovirus, human immunodeficency virus, or herpes simplex virus.
